(19) United States
(12) Patent Application Publication
(10) Pub. No.: US 2026/0000430 A1
(43) Pub. Date: Jan. 1, 2026

(54) INTRAVAGINAL DEVICES

(71) Applicant: PREGEASE INC., Winnipeg (CA)

(72) Inventor: Ayaz Mohammed NOOR MOHAMMED, Winnipeg (CA)

(21) Appl. No.: 19/240,893

(22) Filed: Jun. 17, 2025

Related U.S. Application Data
(60) Provisional application No. 63/664,546, filed on Jun. 26, 2024.

(51) Int. Cl. A61B 17/43

(52) U.S. Cl. CPC A61B 17/43

(57) ABSTRACT

An intravaginal device includes a tubular body extending longitudinally along a body axis between a closed end and an open end axially opposite the closed end, the open end having an opening to an interior of the tubular body for receiving one or more fingers to facilitate insertion and positioning of the tubular body in the vaginal canal with the closed end toward the cervix to facilitate retention of a substance in the vaginal canal. The device further includes a handle fixed to the open end of the body without obstructing the opening to facilitate removal of the body from the vaginal canal while accommodating insertion of the one or more fingers through the opening.

### CROSS-REFERENCE TO RELATED APPLICATION(S)

This application claims the benefit of U.S. Provisional Patent Application No. 63/664,546 filed Jun. 26, 2024, which is hereby incorporated herein by reference in its entirety.

### FIELD

The teachings disclosed herein relate generally to reproductive health, and more specifically, to intravaginal devices for delivery and/or retention of substances within the vaginal canal.

### INTRODUCTION

United States Pat. App. Pub. No. 2004/0089312A1 (Jordan) discloses a fertility plug, for use by both humans and animals, constructed from an impermeable polymeric film membrane covering an elastically deformable, compressible interior, which, because of pressure from the vaginal walls on the unit (forming a seal), prevents escape of semen from a vagina immediately after ejaculation or injection of semen into the vagina, but instead remain for a limited time in the vicinity of the cervix and, thus available for fertilization. A string may be attached to the unit to assist the removal of the plug after the unit has been in the vagina for a sufficient time for sperm to have entered the uterus. A nonspermicidal lubricant can be used with the fertility plug to facilitate insertion into and removal from the vagina; the nonspermicidal lubricant also enhances the seal formed between the unit's surface and the vaginal wall, further restricting the loss of semen from the vagina.

United States Pat. App. Pub. No. 2005/0028824A1 (Jordan et al.) discloses an intravaginal fertility plug, for use by both humans and animals, constructed from regions consisting of impermeable and permeable, polymeric film membrane covering, completely or partially, an elastically deformable and compressible interior, which, because of pressure from the vaginal walls on the unit (forming a seal), can be used to promote fertility by temporarily sealing in semen in the female reproductive tract. A lubricant can be used with the intravaginal fertility plug to facilitate insertion into and removal from the vagina; the lubricant would enhance the seal formed between the unit's surface and the vaginal wall and may contain medication itself.

U.S. Pat. No. 10,849,659B2 (Haeri) discloses a fertility device configured to be placed into a subject's vagina after intercourse, the fertility device comprising: a vaginal portion, the vaginal portion comprising: a proximal end; a distal end opposite the proximal end along a longitudinal axis; and a middle portion between the proximal and the distal end; and a lead operatively coupled to the proximal end, wherein: when at least part of the middle portion is placed into a subject's vagina, the lead remains partially outside the vagina, and the vaginal portion impedes leakage of sperm.

### SUMMARY

The following summary is intended to introduce the reader to various aspects of the applicant's teaching, but not to define any invention.

According to some aspects, an intravaginal conception aid device includes (a) a tubular body extending longitudinally along a body axis between a closed end for insertion into the vaginal canal and an open end axially opposite the closed end for remaining external the vaginal canal. The open end has an opening to an interior of the tubular body for receiving one or more fingers to facilitate insertion and positioning of the tubular body in the vaginal canal with the closed end toward the cervix to retain sperm in proximity thereto post-insemination. The device further includes (b) a handle including an annular base fixed to the open end of the body and extending about the opening to accommodate insertion of the one or more fingers therethrough, and at least one pull fixed to and projecting from the annular base for pulling the tubular body out from the vaginal canal.

In some examples, the base of the handle is formed of a thicker material than that of the body.

In some examples, the at least one pull comprises a pull ring. In some examples, the pull ring has a pair of attachment points spaced circumferentially apart from each other about the body axis and fixed to the base.

In some examples, the at least one pull comprises a plurality of pulls spaced circumferentially apart from each other about the body axis. In some examples, the plurality of pulls comprise a pair of pull rings on opposite sides of the opening, the pull rings having respective finger holes in alignment with each other across the opening.

In some examples, the base of the handle is generally toroidal.

In some examples, the body has a sidewall bounding the interior radially and extending axially along the body axis from the closed end to the open end, the sidewall terminating at the open end in a rim circumscribing the opening and to which the base of the handle is fixed. In some examples, the body has an endwall extending radially inwardly from the sidewall at the closed end, the endwall bounding the interior axially from the closed end.

In some examples, the closed end is concave axially inwardly toward the interior.

In some examples, the body has an exterior surface comprising a plurality of radially projecting ridges spaced axially apart from each other and extending circumferentially about the body axis, at least some of the ridges for interfacing with vaginal rugae to facilitate retention and sealing in the vaginal canal. In some examples, the plurality of ridges comprise a plurality of major ridges and a plurality of minor ridges axially interspersed between the major ridges.

In some examples, the tubular body has a radially expanded portion extending along the body axis to the closed end, a radially constricted portion extending along the axis to the open end, and a tapered portion tapering radially along the body axis from the expanded portion to the constricted portion.

In some examples, the body has an exterior surface comprising a plurality of radially projecting ridges spaced axially apart from each other and extending circumferentially about the body axis, at least some of the ridges for interfacing with vaginal rugae to facilitate retention and sealing in the vaginal canal, and the plurality of radial ridges including a first set of ridges along the expanded portion and a second set of ridges along the constricted portion.

In some examples, an exterior of the body is pre-lubricated to facilitate insertion of the tubular body into the vaginal canal. In some examples, the interior of the body is pre-lubricated to facilitate insertion and removal of the one or more fingers into and out from the interior through the opening.

In some examples, the body is formed of an elastomeric material for elastically conforming to the vaginal canal.

In some examples, the body is of integral, unitary, one-piece construction.

In some examples, the handle is formed of an elastomeric material. In some examples, the handle is of integral, unitary, one-piece construction.

In some examples, the body has an interior surface directed toward the interior, an exterior surface opposite the interior surface, and a thickness between the interior and exterior surfaces of at least 0.4 mm. In some examples, the thickness is generally uniform throughout the body.

An intravaginal device includes: (a) a tubular body extending longitudinally along a body axis between a closed end and an open end axially opposite the closed end, the open end having an opening to an interior of the tubular body for receiving one or more fingers to facilitate insertion and positioning of the tubular body in the vaginal canal with the closed end toward the cervix to facilitate retention of a substance in the vaginal canal; and (b) a handle fixed to the open end of the body without obstructing the opening to facilitate removal of the body from the vaginal canal. In some examples, the handle is formed of a thicker material than that of the body.

### DRAWINGS

For a better understanding of the described examples and to show more clearly how they may be carried into effect, reference will now be made, by way of example, to the accompanying drawings in which:

FIG. 1 is a perspective view taken from one end of an example intravaginal device;

FIG. 2 is another perspective from an opposite end of the device of FIG. 1;

FIG. 3 is a side view of the device of FIG. 1;

FIG. 4 is an enlarged view of a portion of the device of FIG. 3;

FIG. 5 is an enlarged view of another portion of the device of FIG. 3;

FIG. 6 is another side view of the device of FIG. 1;

FIG. 7 is a cross-sectional view of the device of FIG. 1, taken along line 7-7 in FIG. 6;

FIG. 8 is an end view from an open end of the device of FIG. 1;

FIG. 9 is an end view from a closed end of the device of FIG. 1;

FIG. 10 is a perspective view of another example intravaginal device;

FIG. 11 is a side view of the device of FIG. 10;

FIG. 12 is an enlarged view of a portion of the device of FIG. 11;

FIG. 13 is a perspective view of another example intravaginal device;

FIG. 14 is a perspective view of another example intravaginal device;

FIG. 15 is a side view of the device of FIG. 14; and

FIG. 16 is a side view of another example intravaginal device.

### DESCRIPTION OF VARIOUS EXAMPLES

Various devices, systems, or processes will be described below to provide an example of each claimed invention. No example described below limits any claimed invention and any claimed invention may cover processes, systems, or devices that differ from those described below. The claimed inventions are not limited to devices, systems, or processes having all of the features of any one device, system, or process described below or to features common to multiple or all of the devices, systems, or processes described below. It is possible that a device, system, or process described below is not an example of any claimed invention. Any invention disclosed in a device, system, or process described below that is not claimed in this document may be the subject matter of another protective instrument, for example, a continuing patent application, and the applicants, inventors, or owners do not intend to abandon, disclaim, or dedicate to the public any such invention by its disclosure in this document.

Fertility and conception are fundamental aspects of reproductive health that affect many individuals and couples worldwide. Despite advances in reproductive medicine, many couples still face challenges in achieving successful conception. Various factors, including sperm motility, sperm count, and the timing of insemination, play crucial roles in the likelihood of successful conception. Traditional methods of aiding successful conception include intrauterine insemination (IUI) and in vitro fertilization (IVF). These methods can be invasive, expensive, and emotionally taxing.

Another option is the use of intravaginal conception aids designed for insertion into the vagina post-insemination to help retain sperm toward the cervix. By creating a more favorable environment for sperm retention and reducing sperm leakage, these devices aim to enhance the likelihood of successful conception. Some such devices may present challenges in terms of insertion, usage, and/or removal by a user, which in some cases can be uncomfortable or unpleasant and deter use.

Intravaginal devices can also be used to facilitate delivery and/or retention of other substances (e.g. other than sperm) within the vaginal canal. For example, intravaginal devices may also be used to delivery and retain a pharmaceutical substance (e.g. medicament) within the vaginal canal, to promote reliable intravaginal application and/or absorption.

According to aspects of the present disclosure, an intravaginal device is disclosed for insertion into the vagina to retain substances within the vaginal canal. In some examples, the intravaginal device can be used as a conception aid, and is inserted post-insemination to retain sperm in proximity to the cervix. In other examples, the intravaginal device can be used as a drug delivery aid, and is inserted to retain a pharmaceutical substance (e.g. medicament) within the vaginal canal. Some aspects of the device of the present disclosure can help increase comfort, convenience, and/or user-friendliness relative to some other intravaginal device designs, and/or offer an accessible alternative solution for individuals and couples seeking treatment and/or to enhance their fertility naturally.

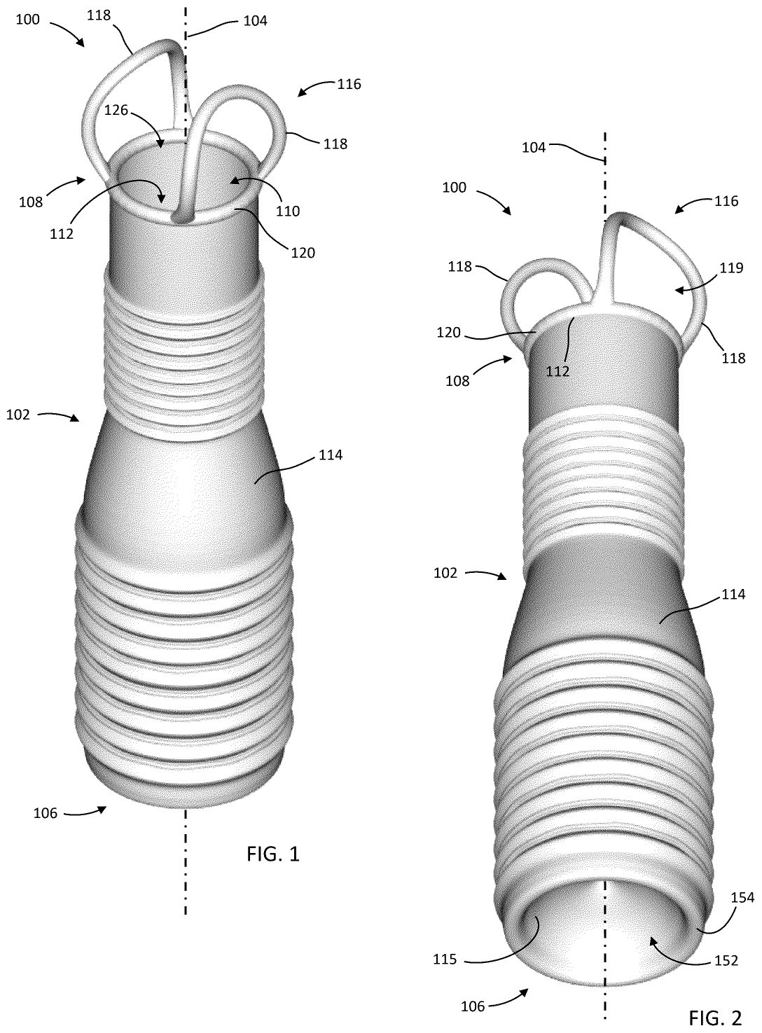

Referring to FIG. 1, an example intravaginal device 100 is shown. In the example illustrated, the device 100 includes a body 102 extending longitudinally along a body axis 104. In the example illustrated, the body 102 is hollow and generally tubular. The body 102 extends along the axis 104 between a closed end 106 for insertion into the vaginal canal and an open end 108 axially opposite the closed end 106 for remaining external the vaginal canal in use. The open end 108 defines an opening 110 to an interior 112 of the tubular body 102. The opening 110 is sized for receiving one or more fingers into the interior 112 from the open end 108 to facilitate insertion and positioning of the tubular body 102 in the vaginal canal with the closed end 106 positioned toward the cervix to help retain a substance in proximity to the cervix. In the example illustrated, the device 100 is configured for retaining semen in proximity to the cervix post-insemination, but may also be used for delivery and/or retention of other substances (e.g. an intravaginal medicament, such as suppositories, tablets, creams, ointments, gels, etc. for treatment of vaginal conditions and/or which may provide therapeutic benefits from application through the vaginal canal). In the example illustrated, the body 102 is formed of an impermeable membrane 113 for blocking escape of substances (e.g. seminal fluid) therethrough, and is elastically conformable to the vaginal canal, which can improve comfort and facilitate sealing under the pressure from the vaginal walls.

In the example illustrated, the device 100 further includes a handle 116 fixed to the open end 108 of the body 102 to assist with removal of the device post-use. The handle 116 is fixed to the open end 108 without obstructing the opening 110 to accommodate insertion of the one or more fingers through the opening 110. In the example illustrated, the handle 116 includes at least one pull 118 projecting from the open end 108 away from the tubular body 102 for remaining external the vagina in use and to facilitate removal of the body 102 from the vaginal canal after use.

In the example illustrated, the handle 116 includes an annular base 120 fixed to the open end 108 of the body 102 and extending about the opening 110 to accommodate insertion of the one or more fingers through the opening 110, and the at least one pull 118 is fixed to and projects from the annular base 120 away from the tubular body 102.

In the example illustrated, the interior 112 is bounded radially by a sidewall 114 of the body 102 extending between the closed and open ends 106, 108 of the body 102. The interior 112 is bounded axially at the closed end 106 by an endwall 115 (FIG. 2) of the body 102. The sidewall 114 and endwall 115 are integrally formed from the impermeable membrane 113. Referring to FIGS. 1 and 2, in the example illustrated, the sidewall 114 extends along the axis 104 from the closed end 106 to the open end 108 of the body and terminates at an open-end rim 124 circumscribing the opening 110 about the body axis 104. In the example illustrated, the base 120 of the handle 116 is fixed to the open-end rim 124 at the open end 108 of the body 102 and has a central aperture 126 passing through the base 120 coaxial with the opening 110. In the example illustrated, the base 120 of the handle 116 is generally toroidal. In the example illustrated, the tubular body 102 is generally symmetrical about the body axis 104, with the opening 110, interior 112, sidewall 114, endwall 115, and base 120 generally coaxial with the body axis 104.

In the example illustrated, the handle 116 has a plurality of pulls 118 spaced apart from each other about the body axis 104 (and about the opening 110). In the example illustrated, the handle 116 has a pair of pulls on opposite sides of the body axis 104 (and opening 110) and in alignment with each other across the opening. Each pull 118 comprises a pull ring defining a finger hole 119 for receiving a finger of the user to help with gripping and pulling out the device 100 from the vaginal canal after use. The finger holes 119 of the pair of pulls 119 are in alignment with each other across the axis 104 (and opening 110), for receiving the finger through both pulls 119 at the same time, which can help distribute the pulling force exerted on the base 120 and open end 108 about the axis 104.

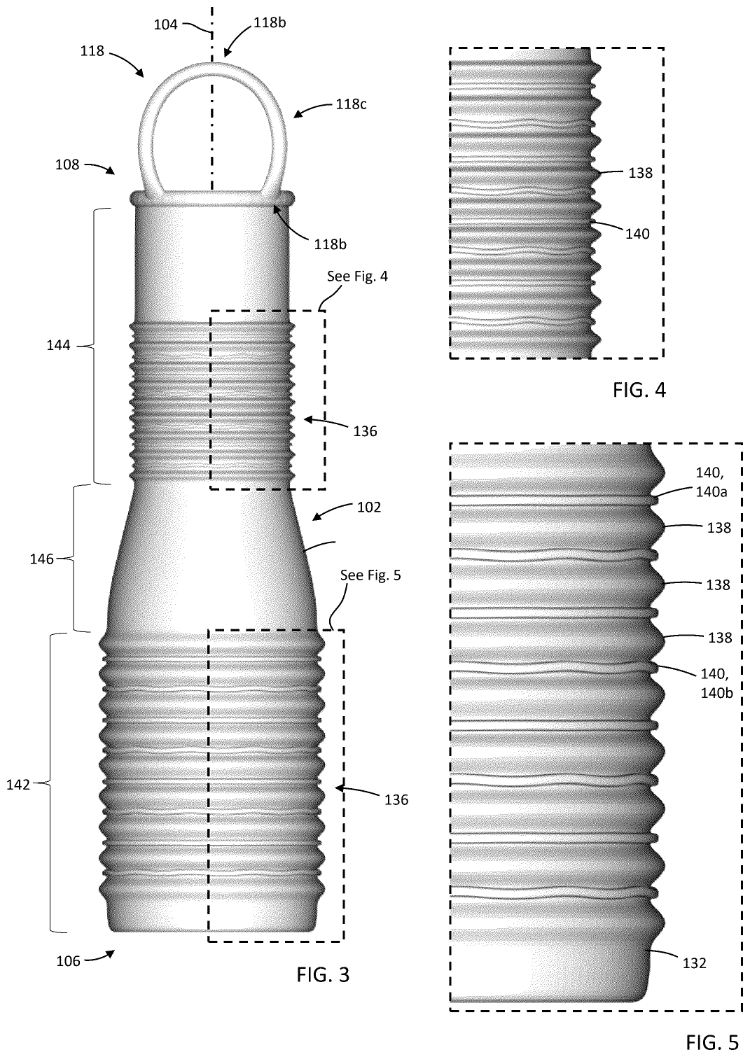

Referring to FIG. 3, in the example illustrated, each pull 118 has a proximal end 118a fixed to the base 120 of the handle 116, a distal end 118b spaced axially apart from the proximal end away from tubular body 102, and a central portion 118c axially intermediate the proximal and distal ends 118a, 118b. In the example illustrated, the proximal end 118a includes a pair of attachment points spaced circumferentially apart from each other about the axis 104 and fixed to the base 120, which can facilitate distribution of the pulling force about the axis 104.

In the example illustrated, the handle 116 is of integral, unitary, one-piece construction, and can be formed of an elastomeric material such as, for example, silicone. The tubular body 102 is formed of an elastomeric material suitable for elastic conformation of the body to the vaginal canal, such as, for example, latex. In the example illustrated, the tubular body 102 is of integral, unitary, one-piece construction. The handle 116 and the body 102 can be formed separately of different materials and subsequently attached.

In some examples, the handle 116 and the body 102 can be of integral, unitary, one-piece construction with each other. In such examples, the same material may be used for both the body 102 and the handle 116 (e.g. latex or silicone). The base 120 of the handle 116 can comprise a reinforced (e.g. thicker) annular portion of the same material circumscribing the opening 110 for keeping the pulls 118 securely attached to the body 102 of the device 100.

Referring to FIG. 7, in the example illustrated, the body 102 has an interior surface 130 directed toward the interior 112 of the body 102 and an exterior surface 132 opposite the interior surface 130. The exterior surface 132 of the body 102 can be pre-lubricated to facilitate insertion of the tubular body 102 into the vaginal canal. The interior surface 130 of the body 102 can be pre-lubricated to facilitate insertion and removal of the one or more fingers through the opening 110. In examples where the device is used as a conception aid, the exterior surface 132 and/or the interior surface 130 of the body 102 can be pre-lubricated with non-spermicidal lubricant. In other examples, the interior surface 130 of the body 102 can be pre-lubricated with any biocompatible lubricant.

In the example illustrated, the body 102 is formed of a membrane material having a thickness 134 between the interior and exterior surfaces 130, 132. The thickness 134 can be generally uniform throughout the body 102, and selected based on the material and shape of the body 102 so that the body 102 is resiliently deformable to generally retain its shape when not in use, while also elastically conforming to the vaginal canal during use under the pressure from the vaginal walls for comfort and improved sealing. The thickness 134 of the body 102 can be, for example, at least 5-15 times greater than that of a typical condom (the thickness of which can typically range from between 0.04 to 0.09 mm). In the example illustrated, the thickness 134 of the body 102 can be at least 0.2 mm. In some examples, the thickness 134 of the body can be at least 0.4 mm. In some examples, the thickness 134 of the body can be at least 0.7 mm. In some examples, the thickness of the body can be at least 0.9 mm.

In the example illustrated, the handle 116 is thicker than the body 102. The handle 116 is formed of a thicker material than that of the body 102. In the example illustrated, the material forming the base 120 of the handle 116 has a base thickness greater than the thickness 134 of the body 102 for reinforcing the connection of the pulls 118 to the body 102. In the example illustrated, the base 120 projects radially outwardly relative to the exterior surface 132 of the body 102 axially adjacent the base 120 (and open end 108). In the example illustrated, the base 120 projects radially inwardly relative to the interior surface 130 of the body 120 axially adjacent the base 120 (and open end 108). The base thickness of the base 120 is defined by a base diameter that is greater than the thickness 134 of the body 102. In the example illustrated, the material forming each pull 118 has a pull thickness greater than the thickness 134 of the body 102. In the example illustrated, the pull thickness of each pull 118 is defined by a pull diameter greater than the thickness 134 of the body 102. The thickness of the handle 116 can be generally uniform, with the base thickness being generally equal to the pull thickness.

Referring to FIG. 3, in the example illustrated, the exterior surface 132 has a plurality of radially projecting ridges 136 spaced axially apart from each other and extending circumferentially about the body axis 104. At least some of the ridges 136 can be sized and arranged for interfacing with vaginal rugae to facilitate retention and sealing in the vaginal canal. Referring to FIG. 5, in the example illustrated, the plurality of ridges 136 include a plurality of major ridges 138 and a plurality of minor ridges 140 axially interspersed between the major ridges 138. In the example illustrated, the major ridges 138 have a major radial extent from the exterior surface 132 of the tubular body 102, and the minor ridges 140 have a minor radial extent from the exterior surface 132. The minor radial extent of the minor ridges 140 is less than the major radial extent of the major ridges 138. The major ridges 138 can be sized and shaped for interfacing with the vaginal rugae, while the minor ridges can also interface with the vaginal canal (e.g. to help improve sealing) and/or be provided to facilitate collapsing of the device 100 along the axis 104—e.g. for packaging, transport, storage, etc. In some examples, the minor ridges 140 can optionally be removed, or replaced with additional major ridges 138.

In the example illustrated, the minor ridges 140 include a set of first minor ridges 140a and a set of second minor ridges 140b axially interspersed with the first minor ridges 140a. In the example illustrated, each first minor ridge 140a extends smoothly and uniformly about the body axis 104 and lies in a respective plane normal to the body axis 104. In the example illustrated, each second minor ridge 140b extends about the tube axis in a sinusoidal (e.g. wave-like) pattern having peaks and troughs along the body axis 104 (i.e. extending upwardly and downwardly relative to a respective plane normal to the body axis 104 and intersecting an axial mid-point of the second minor ridge 140b).

In the example illustrated, the tubular body 102 has a radially expanded portion 142 extending along the body axis 104 from the closed end 106, a radially constricted portion 144 extending along the body axis 104 from the open end 108, and a radially tapered portion 146 tapering radially along the body axis 104 from the expanded portion 142 to the constricted portion 144. Providing the expanded portion 142 at the closed end 106 can help provide an improved seal with the vaginal canal to facilitate retention of substances (e.g. sperm or medicament) toward the cervix. Providing the constricted portion 144 at the open end 108 can help improve comfort during use. In the example illustrated, the body 102 includes a first set of the plurality of ridges 136 along the expanded portion 142 and a second set of the plurality of ridges 136 along the constricted portion 144. The major and minor ridges 138, 140 in the first set of ridges 136 along the expanded portion 142 can have respective radial extents greater than the major and minor ridges 138, 140, respectively, in the second set of ridges along the constricted portion 144.

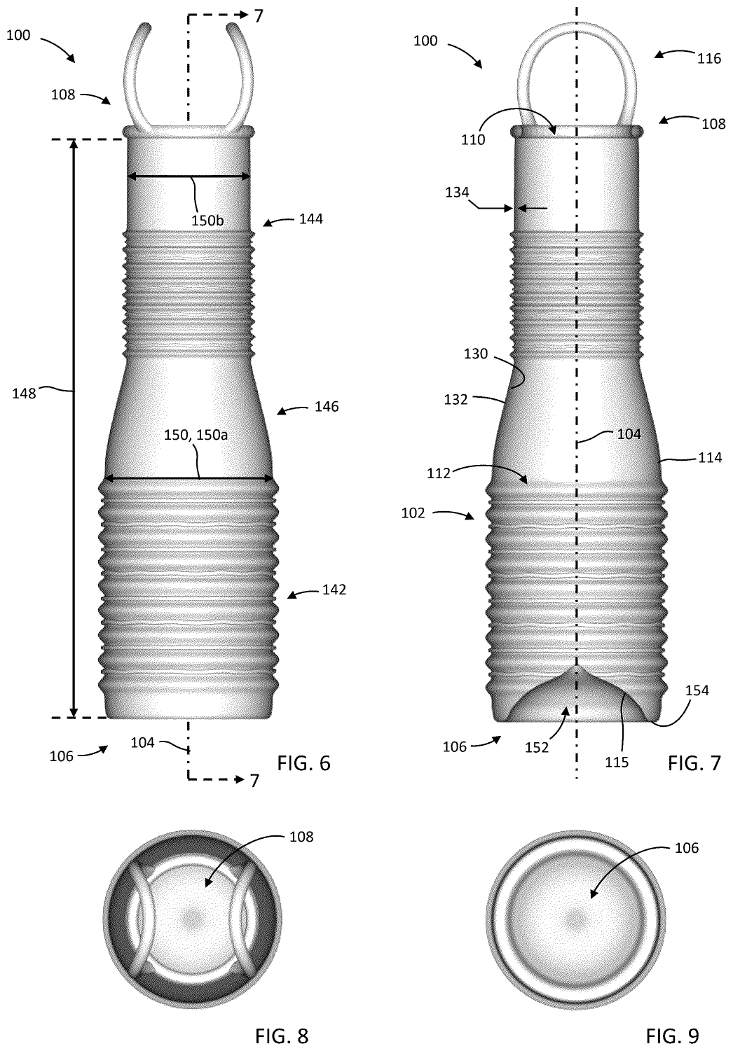

Referring to FIG. 6, the tubular body 102 has a length 148 along the body axis 104 from the closed end 106 to the open end 108. In the example illustrated, the expanded portion 142 extends from the closed end 106 along at least a third of the length 148 of the body 102. The constricted portion 144 extends from the open end 108 along at least a third of the length 148 of the body 102. The tapered portion 146 extends along the axis 104 within a central third of the length 148 of the body 102.

The tubular body 102 has a diameter 150 across the axis 104 between laterally opposite sides of the exterior surface 132. The length 148 and the diameter 150 of the body 102 can be varied to suit different anatomies. In some examples, the length 148 of the body 102 can be around 3.5 to 4.5 inches. The expanded portion 142 has an expanded diameter 150a and the constricted portion 144 has a constricted diameter 150b that is less than the expanded diameter 150a. The expanded diameter 150a at the expanded portion 142 of the body 102 can be around, for example, 2 to 3 inches (and in some examples can be around 2.6 inches). The constricted diameter 150b at the constricted portion 144 of the body 102 can be around, for example, 1.5 to 2.5 inches (and in some examples can be around 1.9 inches).

Referring to FIG. 7, in the example illustrated, the closed end 106 of the body 102 is concave inwardly toward the interior 112, and the exterior surface 132 at the closed end 106 defines a cavity 152 that is open away from the body 102 coaxial with the body axis 104. This can help with, for example, collection, pushing, and/or retention of a substance (e.g. sperm) toward the cervix during insertion of the device 100. In the example illustrated, the sidewall 114 of the body 102 terminates at the closed end 106 in a closed-end rim 154 circumscribing the body axis 104 (and cavity 152), and the endwall 115 of the body 102 extends radially and axially inwardly from the closed-end rim 154 to define the cavity 152. The closed-end rim 154 can comprise a reinforced (e.g. thicker) portion of the body to retain the shape of the cavity 152.

When used as a conception aid, after insemination, a user inserts one or more fingers through the opening 110 at the open end 108 of the body 102 to assist with inserting the closed end 106 into the vaginal canal and sliding the body 102 into position to form a seal around the body 102 through the pressure of the vaginal walls and prevent escape of semen. The handle 116 is left external the vagina. The device 100 remains in position within the vagina temporarily (e.g. for one hour), after which the user can grasp the handle 116 and pull out the device 100.

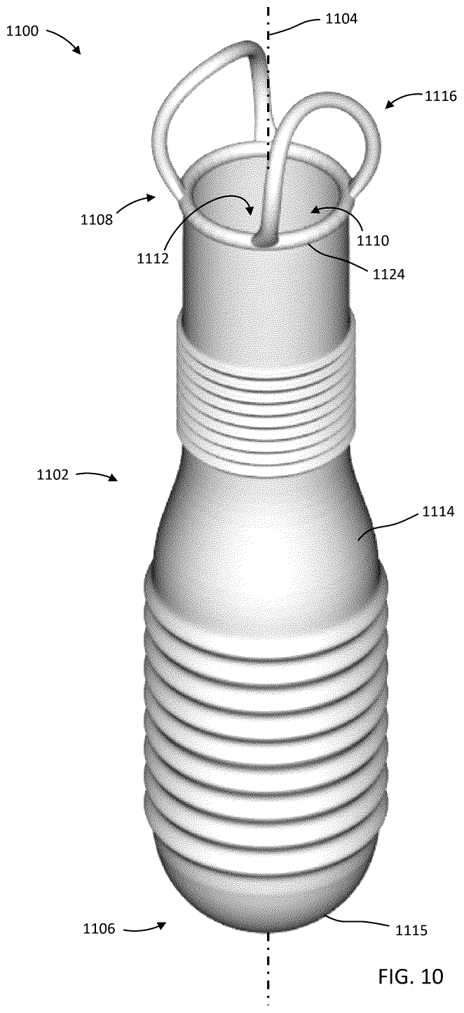

Referring to FIG. 10, another example intravaginal device 1100 is illustrated. The device 1100 has similarities to the device 100 and like features are identified with like reference characters, incremented by 1000.

In the example illustrated, the device 1100 includes a body 1102 extending longitudinally along a body axis 1104 between a closed end 1106 for insertion into the vaginal canal and an open end 1108 axially opposite the closed end 1106. The open end 1108 defines an opening 1110 to an interior 1112 of the body 1102 for receiving one or more fingers to facilitate insertion and positioning of the device 1100. The device 1100 includes a handle 1116 fixed to the open end 1108 of the body 1102 without obstructing the opening 1110 to accommodate insertion of the one or more fingers through the opening 1110.

The body 1102 has a sidewall 1114 bounding the interior 1112 radially and extending along the axis 1104 from the closed end 1106 to the open end 1108. The sidewall 1114 terminates at the open 1108 at an open-end rim 1124 circumscribing the opening 1110 about the body axis 1104. The handle 1116 is fixed to the open-end rim 1124 of the sidewall 1114. In the example illustrated, the body 1102 has an endwall 1115 extending radially inwardly from the sidewall 1114 at the closed end 1106. The endwall 1115 bounds the interior 1112 axially at the closed end 1106. In the example illustrated, the closed end 1106 is convex, with the endwall 1115 projecting axially outwardly away from the interior 1112 of the body 1102, which may facilitate insertion of the device.

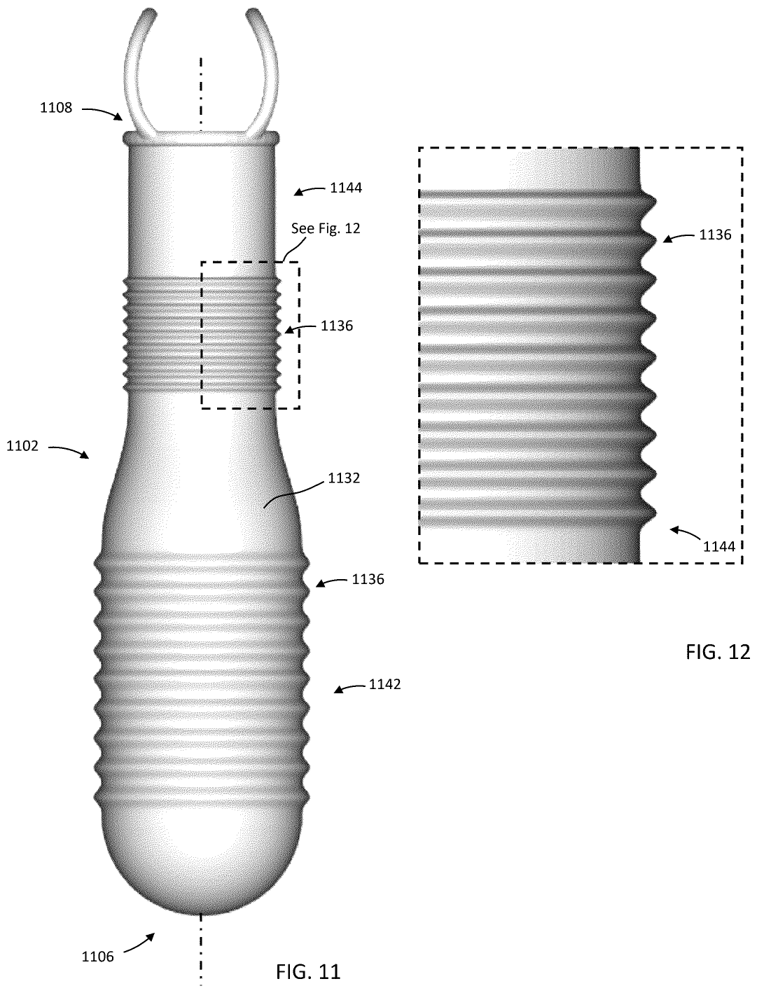

Referring to FIGS. 11 and 12, in the example illustrated, the body 1102 has an exterior surface 1132 with a plurality of radially projecting ridges 1136 spaced axially apart from each other and extending circumferentially about the body axis 1104. One set of the ridges 1136 is provided along a radially expanded portion 1142 of the body 1102, and another along a radially constricted portion 1144 of the body 1102. Referring to FIG. 12, in the example illustrated, the ridges 1136 along at least the constricted portion 1144 have a parallel, pleated (zigzag) configuration configured to facilitate axial collapse of the body 1102 along the plurality of ridges 1136.

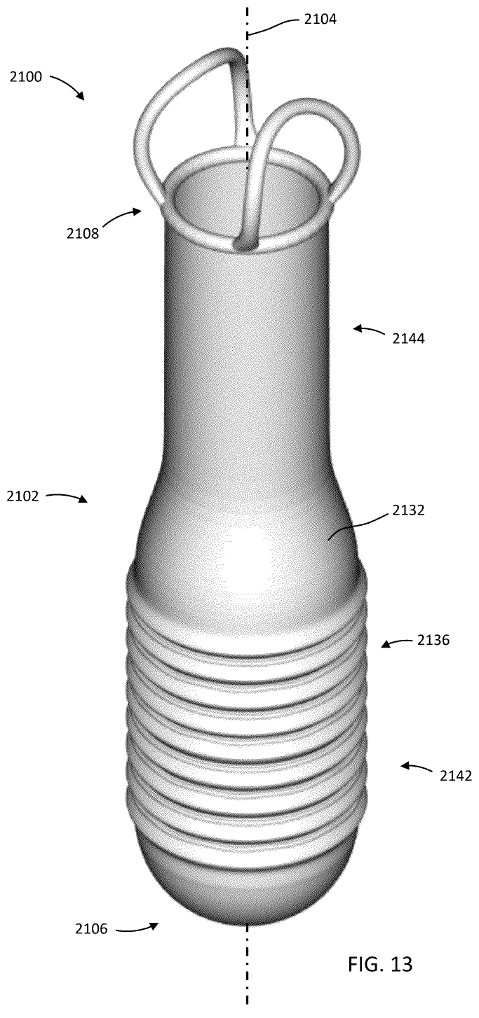

Referring to FIG. 13, another example intravaginal device 2100 is illustrated. The device 2100 has similarities to the device 1100 and like features are identified with like reference characters, incremented by 1000. In the example illustrated, the device 2100 includes a body 2102 extending longitudinally along a body axis 2104 between a closed end 2106 for insertion into the vaginal canal and an open end 2108 axially opposite the closed end 2106. In the example illustrated, the body 2102 has an exterior surface 2132 with a plurality of radially projecting ridges 2136 spaced axially apart from each other and extending circumferentially about the body axis 2104. The ridges 2136 are provided along a radially expanded portion 2142 of the body 2102. The body 2102 has a radially constricted portion 2144, which is free of any ridges 2136 in the example illustrated.

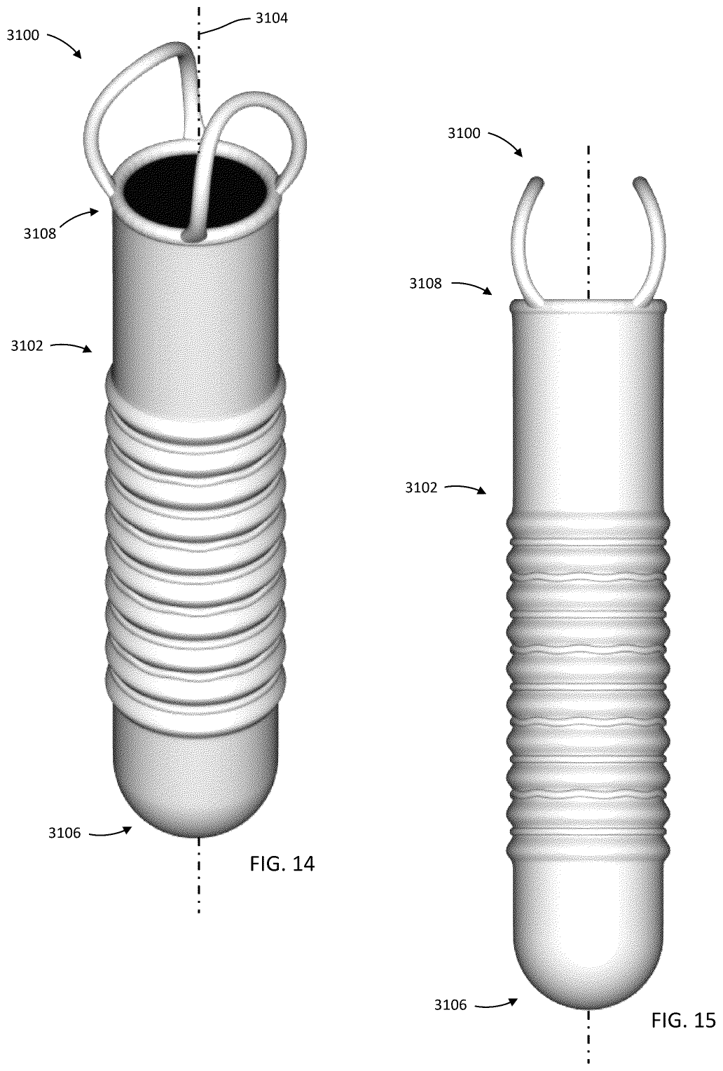

Referring to FIGS. 14 and 15, another example intravaginal device 3100 is illustrated. The device 3100 has similarities to the device 2100 and like features are identified with like reference characters, incremented by 1000. In the example illustrated, the device 3100 includes a body 3102 extending longitudinally along a body axis 3104 between a closed end 3106 for insertion into the vaginal canal and an open end 3108 axially opposite the closed end 3106. In the example illustrated, the body 3102 has a generally constant diameter along its length from the open end 3108 to the closed end 3106.

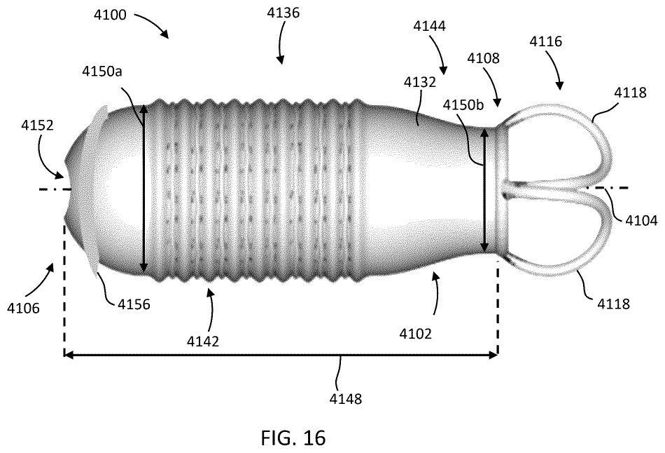

Referring to FIG. 16, another example intravaginal device 4100 is illustrated. The device 4100 has similarities to the device 2100 and like features are identified with like reference characters, incremented by 2000. In the example illustrated, the device 4100 includes a body 4102 extending longitudinally along a body axis 4104 between a closed end 4106 for insertion into the vaginal canal and an open end 4108 axially opposite the closed end 4106.

In the example illustrated, the body 4102 has an exterior surface 4132 with a plurality of radially projecting ridges 4136 spaced axially apart from each other and extending circumferentially about the body axis 4104. The ridges 4136 are provided along a radially expanded portion 4142 of the body 4102. The body 4102 has a radially constricted portion 4144, which is free of any ridges 4136 in the example illustrated.

In the example illustrated, the device is configured for delivery and retention of a pharmaceutical substance (e.g. medicament) in the vaginal canal. The body 4102 has a length 4148, which in the example illustrated, is generally than in the other example devices described herein (e.g. device 100, 2100, etc.). This can allow for application and retention of the medicament in the vaginal canal further from the cervix relative to some of the other devices disclosed herein. The radially constricted portion 4144 of the device 4100 is also shorter in length relative to the radially expanded portion 4142 of the device 4100 in the example illustrated.

In some examples, the length 4148 of the body 4102 can be around 1.5 to 2.5 inches (and in some examples can be around 2 inches). The expanded portion 4142 has an expanded diameter 4150a and the constricted portion 4144 has a constricted diameter 4150b that is less than the expanded diameter 4150a. The expanded diameter 4150a at the expanded portion 4142 of the body 4102 can be around, for example, 1 to 2 inches (and in some examples can be around 1.5 inches).

In the example illustrated, the device 4100 further includes a handle 4116 including at least one pull 4118. In the example illustrated, the handle 4116 includes a pair of pulls 4118. Each pull 4118 comprises a pull ring in the example illustrated. The length of each pull 4118 in the axial direction can be around, for example, 1.5 to 2.5 inches (and in some examples can be around 2 inches).

In the example illustrated, the device 4100 further comprises an annular flap 4156 projecting radially from the body 4102 and extending circumferentially about the body 4102 adjacent the adjacent the closed end 4106 of the device 4100. The flap 4156 can facilitate application, delivery, and retention of the substance at the around the closed end 4106 of the device, and may further help with sealing of the device 4100 in the vaginal canal.

In the example illustrated, the closed end 4106 of the body 4102 comprises a cavity 4152 that is open away from the body 4102 coaxial with the body axis 4104. This can help with, for example, application, collection, pushing, and/or retention of a substance toward the cervix and/or within the vaginal canal during insertion of the device 4100. The cavity 4152 in some examples may be sized and shaped to retain pharmaceutical substances, for example, tablets, liquids, gels, powdered pharmaceutical substances, etc., while the device is being inserted into the vaginal canal.

The device 4100 in the present example is configured for use as a medicament delivery aid. A medicament can be placed within or proximate the cavity 4152 of the device 4100 (e.g. about the closed end 4106 and/or flap 4156) before insertion of the device 4100 into the vaginal canal. The relatively shorter length 4148 of the body 4102 of the device 4100 retains the pharmaceutical substance within the vaginal canal at an intermediate depth further from the cervix relative to other devices disclosed herein (e.g. to facilitate treatment of or absorption through the vaginal canal). The presence of the flap 4156 can assist with the application, retention, and delivery of the substance. The relatively long length of the pulls 4118 allows for the device 4100 to be easily removed, and can serve as a visual indicator for depth positioning of the device 4100 within the vaginal canal. The use of the device 4100 as a drug delivery aid can reduce pharmaceutical substance loss (e.g. leakage out of the vaginal canal), and can maintain the pharmaceutical substance at a predetermined depth, to promote consistent delivery of medicament.

1. An intravaginal conception aid device comprising:
a) a tubular body extending longitudinally along a body axis between a closed end for insertion into the vaginal canal and an open end axially opposite the closed end for remaining external the vaginal canal, the open end having an opening to an interior of the tubular body for receiving one or more fingers to facilitate insertion and positioning of the tubular body in the vaginal canal with the closed end toward the cervix to retain sperm in proximity thereto post-insemination; and
b) a handle including an annular base fixed to the open end of the body and extending about the opening to accommodate insertion of the one or more fingers therethrough, and at least one pull fixed to and projecting from the annular base for pulling the tubular body out from the vaginal canal.

2. The device of claim 1, wherein the base of the handle is formed of a thicker material than that of the body.

3. The device of claim 1, wherein the at least one pull comprises a pull ring.

4. The device of claim 3, wherein the pull ring has a pair of attachment points spaced circumferentially apart from each other about the body axis and fixed to the base.

5. The device of claim 1, wherein the at least one pull comprises a plurality of pulls spaced circumferentially apart from each other about the body axis.

6. The device of claim 5, wherein the plurality of pulls comprise a pair of pull rings on opposite sides of the opening, the pull rings having respective finger holes in alignment with each other across the opening.

7. The device of claim 1, wherein body has a sidewall bounding the interior radially and extending axially along the body axis from the closed end to the open end, the sidewall terminating at the open end in a rim circumscribing the opening and to which the base of the handle is fixed, and the body has an endwall extending radially inwardly from the sidewall at the closed end, the endwall bounding the interior axially.

8. The device of claim 1, wherein the closed end is concave axially inwardly toward the interior.

9. The device of claim 1, wherein the body has an exterior surface comprising a plurality of radially projecting ridges spaced axially apart from each other and extending circumferentially about the body axis, at least some of the ridges for interfacing with vaginal rugae to facilitate retention and sealing in the vaginal canal.

10. The device of claim 9, wherein the plurality of radial ridges comprise a plurality of major ridges and a plurality of minor ridges axially interspersed between the major ridges.

11. The device of claim 1, wherein the tubular body has a radially expanded portion extending along the body axis to the closed end, a radially constricted portion extending along the axis to the open end, and a tapered portion tapering radially along the body axis from the expanded portion to the constricted portion.

12. The device of claim 11, wherein the body has an exterior surface comprising a plurality of radially projecting ridges spaced axially apart from each other and extending circumferentially about the body axis, at least some of the ridges for interfacing with vaginal rugae to facilitate retention and sealing in the vaginal canal, the plurality of radial ridges including a first set of ridges along the expanded portion and a second set of ridges along the constricted portion.

13. The device of claim 1, wherein the interior of the body is pre-lubricated with non-spermicidal lubricant to facilitate insertion and removal of the one or more fingers into and out from the interior through the opening.

14. The device of claim 1, wherein the body is formed of an elastomeric material for elastically conforming to the vaginal canal.

15. The device of claim 1, wherein the body is of integral, unitary, one-piece construction.

16. The device of claim 1, wherein the body has an interior surface directed toward the interior, an exterior surface opposite the interior surface, and a thickness between the interior and exterior surfaces of at least 0.4 mm.

17. The device of claim 16, wherein the thickness is generally uniform throughout the body.

18. The device of claim 1, wherein the handle is of integral, unitary, one-piece construction and formed of an elastomeric material.

19. An intravaginal device comprising:
a) a tubular body extending longitudinally along a body axis between a closed end and an open end axially opposite the closed end, the open end having an opening to an interior of the tubular body for receiving one or more fingers to facilitate insertion and positioning of the tubular body in the vaginal canal with the closed end toward the cervix to facilitate retention of a substance in the vaginal canal; and
b) a handle fixed to the open end of the body without obstructing the opening to facilitate removal of the body from the vaginal canal while accommodating insertion of the one or more fingers through the opening.

20. The device of claim 19, wherein the handle is formed of a thicker material than that of the body.
